(19) United States
(12) Patent Application Publication
(10) Pub. No.: US 2015/0164477 A1
(43) Pub. Date: Jun. 18, 2015

(54) Mobile Ultrasound Diagnosis System Using Two-Dimensional Array Data And Mobile Ultrasound Diagnosis Probe Device And Ultrasound Diagnosis Apparatus For The System

(71) Applicant: HEALCERION CO., LTD., Seoul (KR)

(72) Inventors: Jeong Won Ryu, Seoul (KR); You Chan Choung, Seoul (KR)

(21) Appl. No.: 14/408,062

(22) PCT Filed: Jun. 25, 2013
(86) PCT No.: PCT/KR2013/005578

§ 371 Date: Dec. 15, 2014

(30) Foreign Application Priority Data
Jun. 25, 2012 (KR) 10-2012-0068055

(51) Int. Cl. A61B 8/08; A61B 8/00

(52) U.S. Cl. CPC A61B 8/5207; A61B 8/4427; A61B 8/48

(57) ABSTRACT

Provided is a mobile ultrasound diagnosis system including a mobile ultrasound diagnosis probe device, being portable, digitally processing ultrasonic data obtained from an object, processing digitalized ultrasonic data into two-dimensional array ultrasonic data by adjacently arranging for each ultrasonic frame, and wirelessly transmitting the two-dimensional array ultrasonic data and an ultrasound diagnosis apparatus receiving the two-dimensional array ultrasonic data from the mobile ultrasound diagnosis probe device, decompressing and restoring the two-dimensional array ultrasonic data, and generating ultrasonic image data by compensating a time gain and adjusting brightness and a contrast of the two-dimensional array ultrasonic data.

### TECHNICAL FIELD

The present invention relates to a mobile ultrasound diagnosis system, and more particularly, to a mobile ultrasound diagnosis system processing obtained ultrasonic data into two-dimensional array data to be compressed and wirelessly transmitting the two-dimensional array data to perform ultrasound diagnosis and a mobile ultrasound diagnosis probe device and an ultrasound diagnosis apparatus for the mobile ultrasound diagnosis system.

### BACKGROUND ART

Since having noninvasive and nondestructive properties, ultrasound diagnosis systems are generally used in the medical field to obtain information of the inside of an object. Since high-resolution images of internal organizations of objects may be provided to doctors with no surgical operations of directly incising and observing objects , ultrasound diagnosis systems are very importantly used in the medical field.

Generally, an ultrasound system includes an ultrasound probe, a beam former, a data processor, a scan converter, and a display unit. The ultrasound probe transmits an ultrasonic signal to an object and forms a received signal by receiving a reflected ultrasonic signal, that is, an ultrasonic echo signal. The ultrasound probe includes at least one transducer element operating to convert an ultrasonic signal and an electric signal into one another. The beam former analog/digital-converts the received signal provided from the ultrasound probe, delays a time of a digital signal considering a position and a focusing point of each conversion element, and forms ultrasonic data, that is, radio frequency (RF) data by summing up the time-delayed digital signals. The data processor performs various data processes with respect to ultrasonic data, which are necessary for forming an ultrasonic image. The scan converter scan-converts the processed ultrasonic data to be displayed on a display area of the display unit. The display unit displays the scan-converted ultrasonic data as an ultrasonic image on a screen.

Typically, data processing such as a time gain compensation (TGC) process, a plurality of finite impulse response (FIR) filtering processes, a plurality of decimation processes, an in-phase/quadrature-phase (I/Q) data forming process, and a compression process and a scan conversion are sequentially performed. Due thereto, not only a lot of time is consumed to process a large amount of ultrasonic data but also a frame rate is deteriorated.

### DISCLOSURE OF INVENTION

### Technical Problem

The present invention provides a mobile ultrasound diagnosis system processing ultrasound data obtained from an object into two-dimensional array data to be compressed and wirelessly transmitting the two-dimensional array data to perform ultrasound diagnosis and a mobile ultrasound diagnosis probe device and an ultrasound diagnosis apparatus for the mobile ultrasound diagnosis system.

### Solution to Problem

According to an aspect of the present invention, there is provided a 1. A mobile ultrasound diagnosis system including a mobile ultrasound diagnosis probe device, being portable, digitally processing ultrasonic data obtained from an object, processing digitalized ultrasonic data into two-dimensional array ultrasonic data by adjacently arranging for each ultrasonic frame, and wirelessly transmitting the two-dimensional array ultrasonic data and an ultrasound diagnosis apparatus receiving the two-dimensional array ultrasonic data from the mobile ultrasound diagnosis probe device, decompressing and restoring the two-dimensional array ultrasonic data, and generating ultrasonic image data by compensating a time gain and adjusting brightness and a contrast of the two-dimensional array ultrasonic data.

The mobile ultrasound diagnosis probe device may process reception ultrasonic frames of a series stream into two-dimensional array ultrasonic data by adjacently arranging for each ultrasonic frame unit to be perpendicular.

The ultrasound diagnosis apparatus may determine an ultrasonic wave measurement depth according to an input of a user and may determine a parameter for compensating a time gain and parameters for adjusting the brightness and the contrast, based on the ultrasonic wave measurement depth.

The ultrasound diagnosis apparatus may transmit dummy data for automatically measuring a wireless communication environment and determining sizes of transmission data to the mobile ultrasound diagnosis probe device. Also, the mobile ultrasound diagnosis probe device may receive the dummy data from the ultrasound diagnosis apparatus, may calculate an available band of presently used wireless communication by measuring an amount of time for receiving the dummy data, and may determine sizes of data to be wirelessly transmitted according to the available band.

According to another aspect of the present invention, there is provided a mobile ultrasound diagnosis probe device including a transmission signal forming unit forming a transmission signal for obtaining a frame of an ultrasonic image, an ultrasound probe converting the transmission signal of the transmission signal forming unit into an ultrasonic signal, transmitting the ultrasonic signal to an object, and obtaining analog ultrasonic data reflected from the object, a two-dimensional array processing unit processing the obtained analog ultrasonic data into two-dimensional array ultrasonic data by adjacently arranging for each ultrasonic frame, a compressing unit compressing the two-dimensional array ultrasonic data adjacently arranged for each ultrasonic frame, and a wireless communication unit wirelessly transmitting compressed two-dimensional array ultrasonic data to an ultrasound diagnosis apparatus.

The two-dimensional array processing unit may process reception ultrasonic frames of a series stream into two-dimensional array ultrasonic data by adjacently arranging for each frame unit to be perpendicular.

The mobile ultrasound diagnosis probe device may further include a beam former generating digitalized ultrasonic data from the analog ultrasonic data obtained from the ultrasound probe.

The beam former may generate M number of arrays having N size in the case of using the M number of ultrasonic waves for one ultrasonic image frame and performing N times of sampling when the respective ultrasonic waves are reflected and return from the object.

The two-dimensional array processing unit may generate two-dimensional array data having N×M arrays in the case of using M number of ultrasonic waves for one ultrasonic image frame and performing N times of sampling when the respective ultrasonic waves are reflected and return from the object.

The wireless communication unit may include short-distance wireless communications by using any one of Bluetooth, wireless USB, wireless LAN, WiFi, Zigbee, and Infrared data association (IrDA).

According to still another embodiment of the present invention, there is provided an ultrasound diagnosis apparatus including a decompressing unit wirelessly receiving compressed ultrasonic data from a mobile ultrasound diagnosis probe device and decompressing the ultrasonic data by using the same method as a compression method used by the mobile ultrasound diagnosis probe device, a two-dimensional array processing unit processing the decompressed ultrasonic data into two-dimensional array ultrasonic data by adjacently arranging for each ultrasonic frame, a time gain compensation unit compensating a time gain with respect to the two-dimensional array ultrasonic data, a brightness and contrast adjustment unit adjusting brightness and a contrast with respect to the two-dimensional array ultrasonic data, and a control unit generating an ultrasonic image for diagnosis by using two-dimensional array ultrasonic data having a compensated time gain and adjusted brightness and contrast.

The time gain compensation unit may compensate ultrasonic data according to a time gain compensation table.

The brightness and contrast adjustment unit may change a brightness value of a certain value or less into 0 and may change a brightness value of a certain value or more into a maximum value.

The brightness and contrast adjustment unit may change a contrast value of a certain value or less into 0 and may change a contrast value of a certain value or more into a maximum value.

### Advantageous Effects of Invention

According to the present embodiment, since a processing amount of ultrasonic data may be reduced by two-dimensional data processing operation in a mobile ultrasound diagnosis probe device, it is possible to simplify programs operated in the ultrasound diagnosis apparatus and to reduce an amount of consumed source such as a memory and a central processing unit (CPU). In addition, the ultrasound diagnosis apparatus performs time gain compensation operations and brightness and contrast adjustment operations, thereby providing stability.

Also, the mobile ultrasound diagnosis probe device transmits two-dimensionally arranged ultrasonic data to the ultrasound diagnosis apparatus in such a way that the ultrasound diagnosis apparatus may apply various image processing processes by using original ultrasonic data. DR

### BRIEF DESCRIPTION OF DRAWINGS

FIG. 1 is a block view illustrating a mobile ultrasound diagnosis system according to an embodiment of the present invention;

FIG. 2 is a view illustrating transmission ultrasonic frames of an ultrasonic probe according to an embodiment of the present invention;

FIG. 3 is a view illustrating ultrasonic data, when M number of ultrasonic waves are used and N times of sampling are performed, according to an embodiment of the present invention;

FIG. 4 is a view illustrating two-dimensional arrangement according to an embodiment of the present invention;

FIGS. 5 to 7 are views illustrating a two-dimensional arrangement process according to an embodiment of the present invention;

FIGS. 8 to 11 are views collectively illustrating the two-dimensional arrangement process shown in FIGS. 5 to 7;

FIG. 12 is a view illustrating a time gain compensation operation according to an embodiment of the present invention;

FIG. 13 is a view illustrating brightness adjustment according to an embodiment of the present invention; and

FIG. 14 is a view illustrating contrast adjustment according to an embodiment of the present invention.

### BEST MODE FOR CARRYING OUT THE INVENTION

Hereinafter, the embodiments of the present invention will be described in detail with reference to the attached drawings. The following embodiments are provided as examples to fully convey the spirits of the present invention to a person skilled in the art. Accordingly, the present invention is not limited to the embodiments described below and may be embodied in other forms. Also, in the drawings, a width, a length, and a thickness of an element may be exaggerated for convenience of description. Like reference numerals designate like elements throughout.

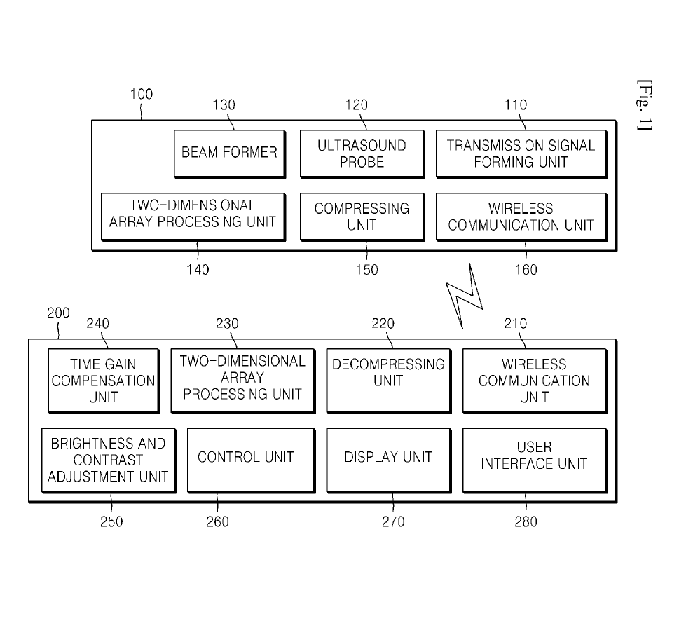

FIG. 1 is a block view illustrating a mobile ultrasound diagnosis system according to an embodiment of the present invention.

Referring to FIG. 1, the ultrasound diagnosis system may include a mobile ultrasound diagnosis probe device 100 and an ultrasound diagnosis apparatus 200.

The mobile ultrasound diagnosis apparatus 100 may include a transmission signal forming unit 110, an ultrasound probe 120 including a plurality of transducer elements, a beam former 130, a two-dimensional array processing unit 140, a compressing unit 150, and a wireless communication unit 160.

The transmission signal forming unit 110 forms a plurality of transmission signals for obtaining a frame of an ultrasonic image by considering the transducer elements and a focusing point of the ultrasound probe 120. A frame is formed of a plurality of scan lines. Also, an ultrasonic image may include a brightness mode (B-mode) image showing a reflection coefficient of an ultrasonic echo signal as a two-dimensional image, a doppler mode (D-mode) image showing a velocity of a moving object as a doppler spectrum by using a Doppler effect, a color mode (C-mode) image showing velocities of a moving object and a scatterer as colors by using the Doppler effect, an elastic mode (E-mode) image showing a mechanical response difference of a medium between when not applying stress on an object and when applying stress on the object as an image, and a three-dimensional mode (3D-mode) image showing a reflection coefficient of an ultrasonic echo signal reflected from an object as a three-dimensional image.

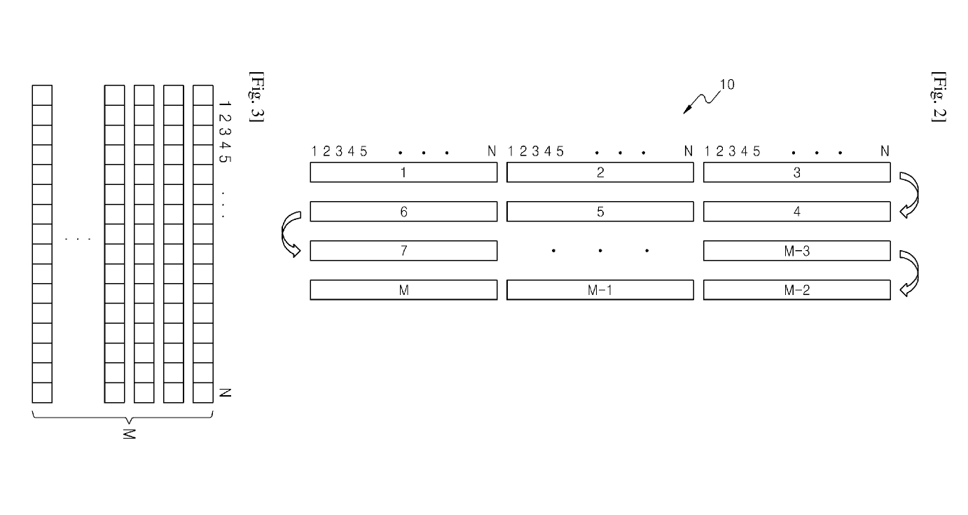

The ultrasound probe 120, as shown in FIG. 2, converts a transmission signal provided from the transmission signal forming unit 110 into an ultrasonic signal and transmits the ultrasonic signal to an object. The ultrasound probe 120 receives an ultrasonic echo signal reflected from the object and forms a reception signal. The ultrasound probe 120 forms a plurality of reception signal by repetitively performing transmission and reception of ultrasonic signals by using a plurality of transmission signals provided from the transmission signal forming unit 110. In this case, the ultrasonic signals transmitted and received by the ultrasound probe 120 have frame data, which are designated as ultrasonic frames. For example, an ultrasonic frame transmitted from the ultrasound probe 120 to a human body is designated as a transmission ultrasonic frame and an ultrasonic frame echoed from the human body to the ultrasound probe 120 is designated as a reception ultrasonic frame.

In the present embodiment, the ultrasound probe 120 may be embodied as a convex probe, a linear probe, a three-dimensional (3D) probe, a trapezoidal probe, and an intravascular ultrasound (IVUS) probe.

The beam former 130 analog/digital-converts a plurality of reception signals provided from the ultrasound probe 120 and generates digitalized ultrasonic data. In addition, the beam former 130 forms a plurality of digital reception focused beams by receiving and focusing the plurality of digitally converted reception signals while considering locations of transducer elements of the ultrasound probe 120 and a focus point. In the present embodiment, the beam former 130 may be embodied as a field programmable gate array or an application specific integrated circuit to improve a speed of processing the reception signals.

Digitalized ultrasonic data, as shown in FIG. 3, is data stored to have an array form that may be shown by a brightness value in an ultrasonic image. A size of an array is determined according to the number of sampling ultrasonic waves reflected and returning from the human body. The number of arrays for one ultrasonic image may be determined according to the number of ultrasonic waves used for one ultrasonic image. In the case of using M number of ultrasonic waves for one ultrasonic image and performing N times of sampling when the respective ultrasonic waves are reflected and return from the human body, the M number of arrays having a size of N may be generated.

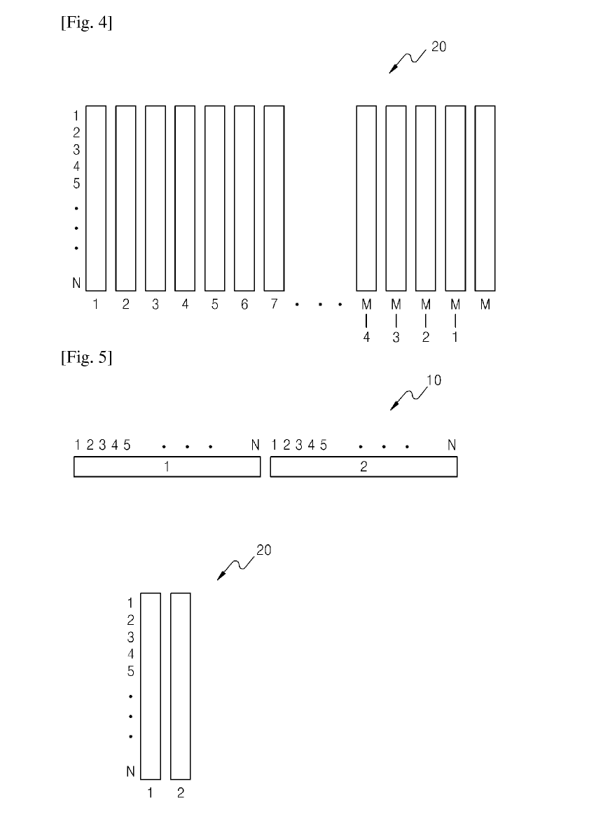

The two-dimensional array processing unit 140 processes ultrasonic data into two-dimensional array ultrasonic data. The two-dimensional array processing unit 140 may form a two-dimensional array 20 as shown in FIG. 4 by adjacently arranging reception ultrasonic frames echoed from the human body.

The two-dimensional array processing unit 140, for example, may adjacently arrange the reception ultrasonic frames echoed from the human body to be perpendicular, instead of forming an image by collecting the reception ultrasonic frames echoed from the human body. The two-dimensional array processing unit 140 provides the respective reception ultrasonic frames disposed adjacently to the compressing unit 150 to compress the same.

As the reception ultrasonic frames echoed from the human body are adjacently arranged instead of being collected and forming an image, continuity of an image pattern is increased and a size of data becomes very smaller relatively to that of image data. When a size of data to be processed becomes smaller, an amount of data to be processed in a compressing process performed by the compressing unit 150 may be reduced.

FIGS. 5 to 7 are views illustrating a two-dimensional arrangement process according to an embodiment of the present invention.

Referring to FIG. 5, the ultrasound probe 120 sequentially sends a first transmission ultrasonic frame and a second transmission ultrasonic frame to the human body. A reference numeral 10 designates transmission ultrasonic frames. In addition, the ultrasound probe 120 receives a first reception ultrasonic frame and a second reception ultrasonic frame echoed from the human body. A reference numeral 20 designates reception ultrasonic frames. The two-dimensional array processing unit 140 adjacently arranges the echoed first reception ultrasonic frame and second reception ultrasonic frame to be perpendicular.

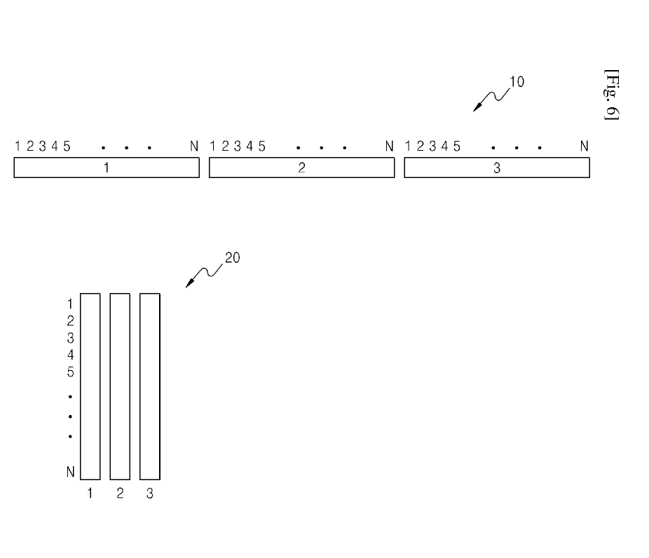

Referring to FIG. 6, the ultrasound probe 120 sends a third transmission ultrasonic frame to the human body. Additionally, the ultrasound probe 120 receives a third reception ultrasonic frame echoed from the human body. The two-dimensional array processing unit 140 adjacently arranges the echoed third reception ultrasonic frame to be perpendicular to the second reception ultrasonic frame.

Referring to FIG. 7, the ultrasound probe 120 sequentially emits an Mth transmission ultrasonic frame to the human body. Additionally, the ultrasound probe 120 receives an Mth reception ultrasonic frame echoed from the human body. The two-dimensional array processing unit 140 adjacently arranges the echoed Mth reception ultrasonic frame to be perpendicular to an (M−1)th reception ultrasonic frame.

In a modified example, the beam former 130 may include a two-dimensional array processing function and may generate an array for storing initial ultrasonic data as a two-dimensional array.

In a modified example, the beam former 130 may include a two-dimensional array processing function and may generate an array for storing initial ultrasonic data as a two-dimensional array.

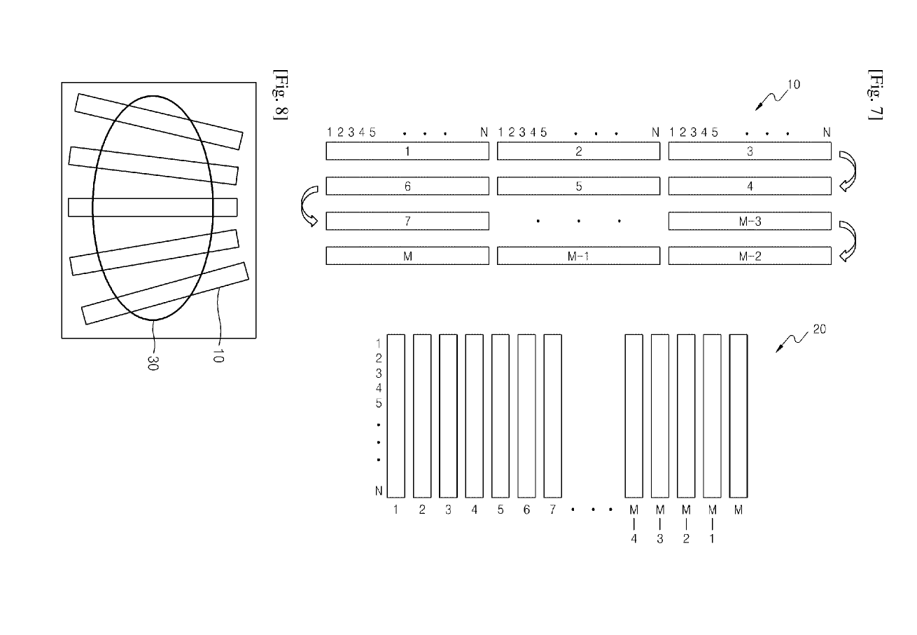

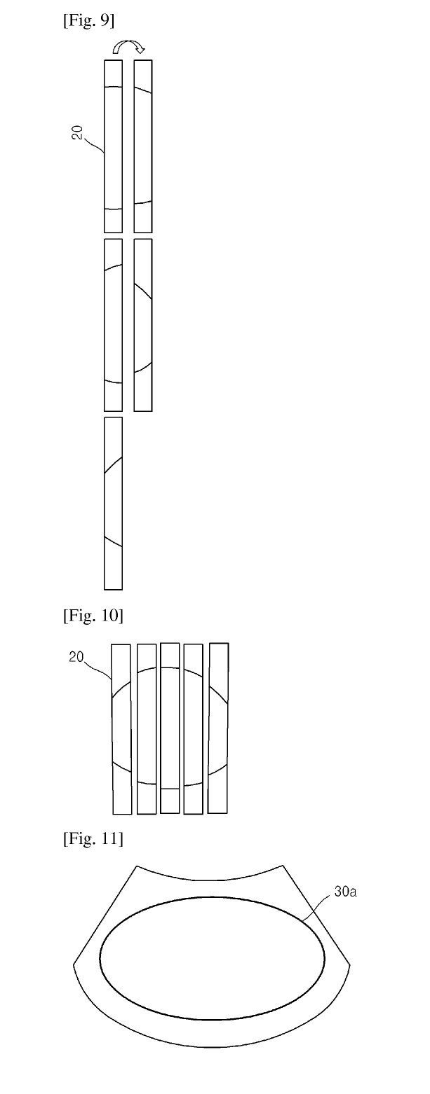

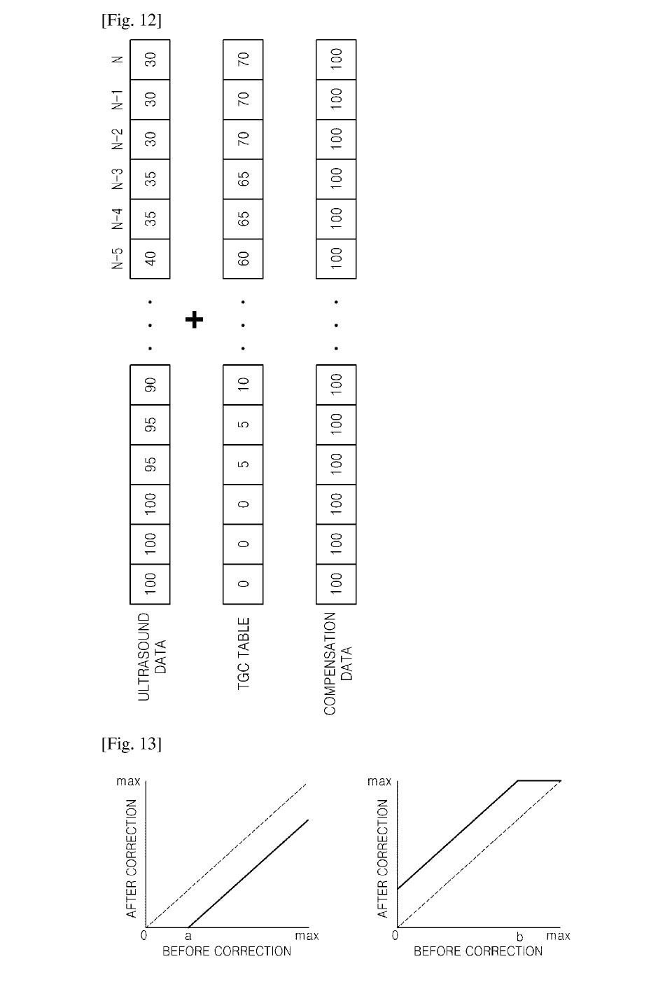

FIGS. 8 to 11 are views for collectively illustrating the two-dimensional arrangement process illustrated in FIGS. 5 to 7. Referring to FIGS. 8 to 11, the ultrasound probe 120 sequentially sends the transmission frames 10 to a human body 30 as shown in FIG. 8. In addition, the ultrasound probe 120 receives the echoed reception ultrasonic frames 20 from the human body 30 as shown in FIG. 9. The two-dimensional array processing unit 140 adjacently arranges the echoed reception ultrasonic frames 20 to be perpendicular and generates a two-dimensional array as shown in FIG. 10. After that, the ultrasonic data generated as the two-dimensional array are transmitted to the ultrasound diagnosis apparatus 200 and form an ultrasonic image 30a for diagnosis, as shown in FIG. 11.

When compressing ultrasonic data into a stream type in which one-dimensional arrays are continuously arranged, since a compression is performed by using only before and behind values in order, a compressibility thereof is not high, which is a reason of applying a two-dimensional array. For example, when using a one-dimensional array, a size of data may be at about 60% of an original size thereof. However, when using image-compression technology by two-dimensionally arranging by using the two-dimensional array processing unit, since all peripheral values may be used, in the case of a compression with no loss, it is possible to compress data to reduce a size at about 30% of an original size thereof. In the case of applying loss compression such as a joint photographic experts group (JPEG) method, a difference therebetween is increased. Also, ultrasonic data two-dimensionally arranged by the two-dimensional array processing unit 140 is transmitted to the ultrasound diagnosis apparatus 200 in such a way that the ultrasound diagnosis apparatus 200 may apply various image processing processes by using original ultrasound data.

The compressing unit 150 compresses ultrasonic data to be transmitted to the ultrasound diagnosis apparatus 200. In order to efficiently use a limited band under wireless communication environments, compression is necessary. The compressing unit 150 compresses two-dimensional array data generated by the two-dimensional array processing unit 140. Accordingly, the compressing unit 150 may increase compressibility by using image compression technology instead of data compression. The compressing unit 150 may use one of compression with no loss and compression with loss, depending on purposes and wireless communication systems.

The wireless communication unit 180 transmits data compressed by the compressing unit 150 to the ultrasound diagnosis apparatus 200.

The wireless communication unit 180 may include short-distance wireless communications by using any one of, for example, Bluetooth, wireless USB, wireless LAN, WiFi, Zigbee, and Infrared data association (IrDA).

The ultrasound diagnosis apparatus 200 may have a wireless communication function and a display device and may include various devices for operating application programs. For example, there may be a personal computer, a smartphone, a tablet type device, a pad type device, and a personal digital assistance (PDA).

The ultrasound diagnosis apparatus 200 may be configured to include a wireless communication unit 210, a decompressing unit 220, a two-dimensional array processing unit 230, a time gain compensation unit 240, a brightness and contrast adjustment unit 250, a control unit 260, a display unit 270, and a user interface unit 280.

The wireless communication unit 210 may include short-distance wireless communications by using any one of, for example, Bluetooth, wireless USB, wireless LAN, WiFi, Zigbee, and IrDA.

The decompressing unit 220 receives ultrasonic data from the mobile ultrasound diagnosis probe device 100 via the wireless communication unit 210.

The decompressing unit 220 obtains two-dimensional array data by decompressing the received ultrasonic data by using the same compression method used by the mobile ultrasound diagnosis probe device 100.

The two-dimensional array processing unit 230 forms an ultrasonic image that can be displayed on a screen of the display unit 220 by using the decompressed two-dimensional array data.

The time gain compensation unit 240 compensates a time gain with respect to the ultrasonic image formed by the two-dimensional array processing unit 230.

Since ultrasonic waves are absorbed in the human body due to properties thereof, ultrasonic waves reflected by a deep part and returning late have great losses and reduced in sizes thereof. In the same human anatomy, a size of ultrasonic data reflected by a deep part has a relatively reduced size. Accordingly, it is necessary to compensate a value increased in proportion to an amount of time for being reflected and returning. When using an ultrasonic data array having N size, a compensation value is determined by generating a time gain compensation table having the same size and then is added to an ultrasonic data array value.

The brightness and contrast adjustment unit 250 adjusts light intensity and a contrast of an ultrasonic image.

When the brightness and contrast adjustment unit 250 decreases brightness values, a brightness value of a certain degree or less is changed into 0. When the brightness and contrast adjustment unit 250 increases brightness values, a brightness value of a certain degree or more is changed into a maximum value.

Accordingly, referring to FIG. 13, when brightness values are decreased by brightness value control operations of the brightness and contrast adjustment unit 250, a brightness value smaller than a is changed into 0. When brightness values are increased, a brightness value greater than b is changed into a maximum value.

The brightness and contrast adjustment unit 250 controls the contrast of an ultrasonic image. When the brightness and contrast adjustment unit 250 adjusts the contrast, a contrast in a bright area having significance in an ultrasonic image may be emphasized and other areas may be one of 0 and a maximum value.

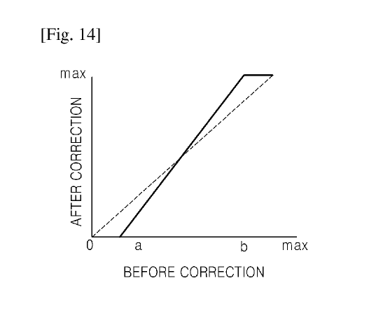

Accordingly, while the brightness and contrast adjustment unit 250 is adjusting the contrast, as shown in FIG. 14, a contrast difference increases when a brightness value is within a range from a to b, the contrast difference is changed into 0 when the brightness value is smaller than a, and the contrast difference is changed into a maximum value when the brightness value is greater than b.

Frequently, ultrasonic data is changed into one of 0 and a maximum values due to operations of the time gain compensation unit 240 and the brightness and contrast adjustment unit 250.

The control unit 260 generates an ultrasonic image by using two-dimensional array ultrasonic data having a compensated time gain and controlled brightness and contrast and allows the ultrasonic image to be displayed on the display unit 270.

In this case, the control unit 260 determines a size of the ultrasonic data by considering a size of the screen of the display unit 270.

The control unit 260 may determine an ultrasonic wave measurement depth according to an input of a user, may determine a parameter used by the time gain compensation unit 240 based on the ultrasonic wave measurement depth, and a degree of adjustment of the brightness and contrast adjustment unit 250.

The control unit 260 may receive the input of the user via the user interface unit 280 and may transmit the input to the ultrasound diagnosis probe device 100 by using wireless communications. For example, the control unit 260 may transmit the ultrasonic wave measurement depth determined to control the mobile ultrasound diagnosis probe device 100 to the mobile ultrasound diagnosis probe device 100.

The control unit 260 may automatically measure a wireless communication environment and may determine sizes of transmission data. The control unit 260 transmits dummy data having a certain size to the mobile ultrasound diagnosis probe device 100.

Accordingly, the wireless communication unit 160 of the mobile ultrasound diagnosis probe device 100 receives the dummy data from the ultrasound diagnosis apparatus 200 and then calculates an available band of presently used wireless communication by measuring an amount of time for receiving the dummy data.

The wireless communication unit 160 of the mobile ultrasound diagnosis probe device 100 determines a size of data to be wirelessly transmitted according to an available band. The smaller a band is, the more reduced a rate of a frame to be transmitted is.

While this invention has been particularly shown and described with reference to preferred embodiments thereof, it will be understood by those skilled in the art that various changes in form and details may be made therein without departing from the spirit and scope of the invention as defined by the appended claims. Therefore, the scope of the invention is defined not by the detailed description of the invention but by the appended claims, and all differences within the scope will be construed as being included in the present invention.

1. A mobile ultrasound diagnosis system comprising:
a mobile ultrasound diagnosis probe device, being portable, digitally processing ultrasonic data obtained from an object, processing digitalized ultrasonic data into two-dimensional array ultrasonic data by adjacently arranging for each ultrasonic frame, and wirelessly transmitting the two-dimensional array ultrasonic data; and
an ultrasound diagnosis apparatus receiving the two-dimensional array ultrasonic data from the mobile ultrasound diagnosis probe device, decompressing and restoring the two-dimensional array ultrasonic data, and generating ultrasonic image data by compensating a time gain and adjusting brightness and a contrast of the two-dimensional array ultrasonic data.

2. The mobile ultrasound diagnosis system of claim 1, wherein the mobile ultrasound diagnosis probe device processes reception ultrasonic frames of a series stream into two-dimensional array ultrasonic data by adjacently arranging for each ultrasonic frame unit to be perpendicular.

3. The mobile ultrasound diagnosis system of claim 1, wherein the ultrasound diagnosis apparatus determines an ultrasonic wave measurement depth according to an input of a user and determines a parameter for compensating a time gain and parameters for adjusting the brightness and the contrast, based on the ultrasonic wave measurement depth.

4. The mobile ultrasound diagnosis system of claim 1, wherein the ultrasound diagnosis apparatus transmits dummy data for automatically measuring a wireless communication environment and determining sizes of transmission data to the mobile ultrasound diagnosis probe device, and
wherein the mobile ultrasound diagnosis probe device receives the dummy data from the ultrasound diagnosis apparatus, calculates an available band of presently used wireless communication by measuring an amount of time for receiving the dummy data, and determines sizes of data to be wirelessly transmitted according to the available band.

5. A mobile ultrasound diagnosis probe device comprising:
a transmission signal forming unit forming a transmission signal for obtaining a frame of an ultrasonic image;
an ultrasound probe converting the transmission signal of the transmission signal forming unit into an ultrasonic signal, transmitting the ultrasonic signal to an object, and obtaining analog ultrasonic data reflected from the object;
a two-dimensional array processing unit processing the obtained analog ultrasonic data into two-dimensional array ultrasonic data by adjacently arranging for each ultrasonic frame;
a compressing unit compressing the two-dimensional array ultrasonic data adjacently arranged for each ultrasonic frame; and
a wireless communication unit wirelessly transmitting compressed two-dimensional array ultrasonic data to an ultrasound diagnosis apparatus.

6. The mobile ultrasound diagnosis probe device of claim 5, wherein the two-dimensional array processing unit processes reception ultrasonic frames of a series stream into two-dimensional array ultrasonic data by adjacently arranging for each frame unit to be perpendicular.

7. The mobile ultrasound diagnosis probe device of claim 5, further comprising a beam former generating digitalized ultrasonic data from the analog ultrasonic data obtained from the ultrasound probe.

8. The mobile ultrasound diagnosis probe device of claim 7, wherein the beam former generates M number of arrays having N size when using the M number of ultrasonic waves for one ultrasonic image frame and performing N times of sampling when the respective ultrasonic waves are reflected and return from the object.

9. The mobile ultrasound diagnosis probe device of claim 5, wherein the two-dimensional array processing unit generates two-dimensional array data having N×M arrays in the case of using M number of ultrasonic waves for one ultrasonic image frame and performing N times of sampling when the respective ultrasonic waves are reflected and return from the object.

10. The mobile ultrasound diagnosis probe device of claim 5, wherein the wireless communication unit comprises short-distance wireless communications by using any one of Bluetooth, wireless USB, wireless LAN, WiFi, Zigbee, and Infrared data association (IrDA).

11. An ultrasound diagnosis apparatus comprising:
a decompressing unit wirelessly receiving compressed ultrasonic data from a mobile ultrasound diagnosis probe device and decompressing the ultrasonic data by using the same method as a compression method used by the mobile ultrasound diagnosis probe device;
a two-dimensional array processing unit processing the decompressed ultrasonic data into two-dimensional array ultrasonic data by adjacently arranging for each ultrasonic frame;
a time gain compensation unit compensating a time gain with respect to the two-dimensional array ultrasonic data;
a brightness and contrast adjustment unit adjusting brightness and a contrast with respect to the two-dimensional array ultrasonic data; and
a control unit generating an ultrasonic image for diagnosis by using two-dimensional array ultrasonic data having a compensated time gain and adjusted brightness and contrast.

12. The ultrasound diagnosis apparatus of claim 11, wherein the time gain compensation unit compensates ultrasonic data according to a time gain compensation table.

13. The ultrasound diagnosis apparatus of claim 11, wherein the brightness and contrast adjustment unit changes a brightness value of a certain value or less into 0 and changes a brightness value of a certain value or more into a maximum value.

14. The ultrasound diagnosis apparatus of claim 11, wherein the brightness and contrast adjustment unit changes a contrast value of a certain value or less into 0 and changes a contrast value of a certain value or more into a maximum value.
